(19) United States
(12) Patent Application Publication
(10) Pub. No.: US 2005/0048015 A1
(43) Pub. Date: Mar. 3, 2005

(54) Cosmetic composition for hair treatment

(76) Inventor: Emile Orenga, Chindrieux (FR)

Correspondence Address: OLIFF & BERRIDGE, PLC, P.O. BOX 19928, ALEXANDRIA, VA, 22320, US

(21) Appl. No.: 10/495,971

(22) PCT Filed: Nov. 27, 2002
(86) PCT No.: PCT/FR02/04071

(30) Foreign Application Priority Data
Nov. 27, 2001 (FR) 01/15563

(51) Int. Cl. A61K 7/09; A61K 7/06

(52) U.S. Cl. 424/70.5; 424/74

(57) ABSTRACT

The invention relates to a cosmetic composition for treating body hair, including the hair, and hair follicles, based on lead acetate and sulfur, in an aqueous-alcoholic solvent containing glycerol, characterized in that it comprises panthenol and a mixture of at least three essential oils chosen from a Saint-Thomas bay essential oil, a ylang-ylang essential oil and a cedarwood essential oil.

The invention relates to a cosmetic composition for treating the hair, but also body hair and hair follicles, intended to recolor white hair via a pigmentation mechanism. This composition is based on lead acetate and sulfur, in an aqueous-alcoholic solvent containing glycerol.

In order to recolor white hair, coloring or dyeing is conventionally carried out. For this, oxidation dyes are used. These are hair-fiber dyes which form when certain strong oxidizing compounds are used to obtain the desired color. Mainly found, for example, are compounds of the arylamine type with derivatives such as para-tolylenediamine (PTDA) or para-phenylenediamine (PPDA) which are combined with couplers of the aminophenol or nitrophenol type in order to modify the coloration. Oxidation with hydrogen peroxide in the presence of aqueous ammonia is then carried out. The problem with these dyes is the unsightly growth of un-dyed roots, not to mention the fact that the hair is damaged due to the severe conditions of the dyeing. It is then necessary to perform a further coloration in order to hide these unsightly white roots. In addition, these diamines appear to cause allergic reactions.

Another process exists for dyeing the hair. This involves lead acetate and, optionally, sulfur and makes it possible to give white hair a natural color again. This treatment, by application of a hair lotion based on lead acetate and, optionally, sulfur, involves the following two mechanisms:
a first which lies in a reaction between the sulfur of the keratin in the hair and the lead salts of the lotion, and
a second which lies in a reaction consisting in absorption of the lead salts from the lotion by the hair, generating lead sulfide or oxides, which are blackish-brown in color.

These two mechanisms result in the depositing of a colored pigment on any hair shaft, thereby recreating hair which appears to have a natural color. The lotion must first be applied daily for a period of 20 to 30 days, and it is then applied regularly, two to three times a week, to maintain the color.

These lead acetate-based hair lotions have the advantage of acting gradually. In fact, after application of the lotion to the hair, the reaction between the lead acetate and the sulfur in the keratin is slow and the lead salts are gradually fixed on the cuticle and in the cortex of the hair.

However, in repeated applications, these compositions have side effects which can become severe. They lead to very rapid degradation of the cuticle of the hair shaft, revealing the cortex which, due to the loss of its natural protection, is exposed to attacks. The hair then becomes dry and brittle, loses its flexibility and is difficult to style. They can even cause a disturbance of the sebaceous glands and the scalp.

The aim of the invention is to produce a hair lotion, intended for the hair in particular, but also for body hair and for hair follicles, which acts according to the same process but which, while conserving the gradual effectiveness, does not have the disadvantages thereof.

Document GB-1 283 892 discloses a hair composition for regenerating the color of the hair, comprising, in an aqueous-alcoholic solution, 1.5% by weight of lead acetate, 1% by weight of sulfur, 0.5% by weight of benzalconium chloride and 2% by weight of a mucopolysaccharide, the incorporation of the latter being intended to improve the appearance of the treated hair. The introduction of this ingredient does not, in any case, make it possible to limit the amount of any one of the active principles responsible for the degradation of the hair, which remains significant.

The applicant has discovered that it is possible to increase the effectiveness of the compositions based on lead acetate and sulfur which are currently available, while at the same time decreasing the amounts of these active principles, and to eliminate the major fault thereof, namely drying out of the treated hair. The applicant has achieved such a composition by adding panthenol and a mixture of essential oils.

Treatment of the hair, body hair and/or hair follicle with a composition of the invention visibly increases the quality of the hair or body hair which has been colored.

In addition to the abovementioned advantages, the introduction of the essential oils makes it possible to eliminate the strong and unpleasant odor of the base ingredients.

Thus, a subject of the invention is a cosmetic composition for treating the hair, body hair and hair follicles, based on lead acetate and sulfur, in an aqueous-alcoholic solvent containing glycerol, said composition comprising panthenol (or vitamin B5) and a mixture of at least three essential oils chosen from a Saint-Thomas bay essential oil, a ylang-ylang essential oil and a cedarwood essential oil.

The expression “aqueous-alcoholic solvent containing glycerol” is intended to mean a mixture of water, alcohol, preferably ethanol, and glycerol. The term “glycerol” is intended to mean a fatty alcohol obtained by saponification of a fatty substance, preferably synthetic or of plant origin, conventionally used in cosmetic moisturizing compositions. The proportion by weight of alcohol in the composition of the invention is preferably greater than 10% so as to allow homogeneous mixing of the various ingredients, but should not reach proportions which are too great, so as to avoid the composition becoming irritant. The proportion of glycerol is preferably greater than or equal to 1.5%, and less than or equal to 3%.

According to an advantageous composition, the proportion by weight of lead acetate relative to the total weight of the composition ranges from 0.10 to 0.60%, preferably from 0.25 to 0.60%, and/or the proportion by weight of sulfur relative to the total weight of the composition ranges from 0.10 to 0.50%, preferably from 0.25 to 0.50%. It is observed that these amounts are much lower than those recommended in the prior art. As indicated above, the composition described in GB-1 283 892 1.5% by weight of lead acetate and 1% by weight of sulfur.

The proportion by weight of panthenol relative to the total weight of the composition preferably ranges from 0.1 to 4%, or better still from 0.4 to 2%.

A composition of the invention as defined above can also comprise at least one activator of the oxidation reaction. This is advantageously chosen from ammonium acetate and ammonium chloride. An optimum proportion by weight of said activator is approximately 0.10% relative to the total weight of the composition, if it is ammonium acetate, and approximately 0.40% relative to the total weight of the composition if it is ammonium chloride.

A composition of the invention as defined above can also comprise at least one other essential oil, preferably chosen from the following oils: rosemary essential oil, lavender essential oil and sage essential oil.

The action of a composition of the invention can also be completed by means of an anti-dandruff action, an anti-seborrheic action, a vitalizing action and/or a hair-styling action. To this effect, a composition advantageously comprises at least one agent capable of conferring on the cosmetic composition anti-dandruff and/or anti-seborrheic and/or vitalizing and/or hair-styling properties.

Thus, in addition to its recoloring effect on white hair, a composition of the invention improves the quality of the hair, in particular from the point of view of its hold, its sheen, its vitality, and ease of styling; it also improves the condition of the scalp and is therefore recommended for problems of dandruff and of seborrhea.

An increase in the rate of coloration of the hair compared to the known compositions is also observed. Thus, at the beginning of treatment, a composition of the invention will preferably be applied daily, for approximately 20 to 30 days, and then, to maintain the color, it will be applied approximately two to three times a week, or even less, since, according to the invention, a coloration is obtained more rapidly.

A composition of the invention may be provided in various forms, such as a lotion, a gel, a milk, a cream or a mousse.

The proportions by weight of the various ingredients of a composition of the invention, relative to the total weight of the composition, are preferably as follows:
lead acetate: 0.1 to 0.6%, preferably from 0.25 to 0.60%, an advantageous proportion being approximately 0.3%
panthenol: 0.1 to 4%, preferably 0.4 to 2%, an advantageous proportion being approximately 0.5%
essential oils: a preferred mixture comprises:
   from 0.02 to 0.2% of Saint-Thomas bay (Pimenta racemosa) essential oil, preferably 0.05%;
   from 0.02 to 0.2% of ylang-ylang (Cananga odorata) essential oil, preferably 0.03%;
   from 0.02 to 0.2% of cedarwood (Cedrus atlantica) essential oil, preferably 0.02%;
   from 0.02 to 0.2% of rosemary (Rosmarinus officinalis) essential oil, preferably 0.03%;
   from 0.02 to 0.2% of lavender (Lavandula angustifolia) essential oil, preferably 0.02%;
   from 0.02 to 0.2% of sage (Salvia officinalis) essential oil, preferably 0.02%.

To obtain a composition of the invention, a mixture of the following ingredients is prepared at ambient temperature: water, alcohol, glycerol, panthenol and essential oils. The solution obtained is then filtered in order to remove the insoluble compounds and possible contaminants. The lead acetate and the sulfur are then added at ambient temperature, with stirring, preferably 3000 rpm.

Of course, other ingredients can be incorporated in order to broaden the properties of a composition of the invention, for example other essential oils which are acknowledged to be active on the properties of the hair and of the scalp or which improves the fragrances of the cosmetic composition.

The characteristics and advantages of the invention compared to the existing lotions are illustrated by the examples below.

### EXAMPLE 1

### Formulation of a Composition of the Invention

This example provides a formulation of a preferred composition of the invention.

| INGREDIENTS: | % w/w |
|---|---|
| Water | qs 100 |
| Alcohol | 14.81 |
| Glycerol | 2.00 |
| Panthenol | 0.50 |
| Sulfur | 0.33 |
| Lead acetate | 0.30 |
| Pimenta racemosa | 0.05 |
| Cananga odorata | 0.03 |
| Rosmarinus officinalis | 0.03 |
| Cedrus atlantica | 0.02 |
| Lavandula angustifolia | 0.02 |
| Salvia officinalis | 0.02 |
| Ammonium acetate | 0.10 |
| Ammonium chloride | 0.40 |

Examples 2 to 4 below demonstrate the role of the ingredients of a composition of the invention, by comparison of the results obtained, in terms of recoloration and of condition of the hair, between various lotions whose formulation is given in examples 2 to 4 and a conventional hair lotion corresponding to the following formulation:

| INGREDIENTS: | % w/w |
|---|---|
| Aqueous-alcoholic water containing glycerol | qs 100 |
| Lead acetate | 0.60-1.50 |
| Sulfur | 0.60-1.50 |
| Benzalconium chloride | 0.10-1.00 |
| Ammonium chloride | 0.50-1.00 |
| Ammonium acetate | 0.10-1.00 |

The results are observed after treatment for 28 days, with daily application.

### EXAMPLE 2

The composition formulated below contains neither benzalconium nor panthenol nor essential oils.

| INGREDIENTS: | % w/w |
|---|---|
| Aqueous-alcoholic water containing glycerol | qs 100 |
| Lead acetate | 0.30 |
| Sulfur | 0.30 |

Compared to the conventional lotion above, a notable loss of the recoloring effect, of the order of 30%, is observed, with a slight improvement in the drying out of the hair.

It is therefore observed that a decrease in the amounts of sulfur and of lead acetate to values falling within the range of those stated according to the present invention and elimination of the benzalconium chloride result in a decrease in the effectiveness of the composition. On the other hand, they limit the drying out of the hair.

### EXAMPLE 3

The composition formulated below contains neither benzalconium nor essential oils, but it does contain panthenol.

| INGREDIENTS: | % w/w |
|---|---|
| Aqueous-alcoholic water containing glycerol | qs 100 |
| Lead acetate | 0.30 |
| Sulfur | 0.30 |
| Panthenol | 0.50 |

Compared to the conventional lotion above, a loss of the recoloring effect, of the order of 15%, is observed, with slight drying out of the hair and an improvement in its texture.

Since the amounts of sulfur and of lead acetate are identical to those in example 2, it is observed that the panthenol has a revitalizing action on the hair, but especially that, in terms of coloration effectiveness, it makes it possible to compensate for the decrease in the amounts of lead acetate and of sulfur. An unexpected role for the panthenol is thus observed.

### EXAMPLE 4

The composition formulated below is a composition of the invention. Compared to a conventional composition, it does not contain any benzalconium, the amounts of lead acetate and of sulfur are decreased, and it comprises panthenol and a mixture of Saint-Thomas bay, ylang-ylang, cedarwood, lavender, rosemary and sage essential oils, according to a formulation of a preferred composition of the invention.

| INGREDIENTS: | % w/w |
|---|---|
| Aqueous-alcoholic water containing glycerol | qs 100 |
| Lead acetate | 0.30 |
| Sulfur | 0.30 |
| Panthenol | 0.50 |
| Saint-Thomas bay | 0.40 |
| Rosemary Cineole BIO* | 0.30 |
| Ylang-Ylang BIO* | 0.30 |
| Cedarwood Atlantica BIO* | 0.20 |
| Lavender Fine | 0.20 |
| Sage officinale BIO* | 0.20 |

*BIO signifies that the essential oil orginates from a biological culture guaranteed to be nonirradiated, pesticide-free and herbicide-free.

A complete recoloration obtained from 20 to 23 days more rapidly than with the conventional lotion is observed, with a notable improvement in the quality of the hair, complete elimination of drying out of the hair and complete revitalizing of the hair with the disappearance of dandruff and of seborrhea.

It emerges from these examples that, compared to a conventional lotion, a composition of the invention makes it possible to recolor the hair more rapidly, while at the same time containing greatly reduced amounts of lead acetate and of sulfur, and being free of benzalconium chloride. It is observed, surprisingly, that decreasing the active principles does not affect the effectiveness of the composition and that this decrease is precisely compensated for by the panthenol and the mixture of essential oils. Since the aggressive constituents are present in more limited amounts and are in part replaced with revitalizing constituents, the quality of the treated hair is considerably improved.

1. A cosmetic composition for treating the hair, body hair and hair follicles, based on lead acetate and sulfur, in an aqueous-alcoholic solvent containing glycerol, characterized in that it comprises panthenol and a mixture of at least three essential oils chosen from a Saint-Thomas bay essential oil, a ylang-ylang essential oil and a cedarwood essential oil.

2. The composition as claimed in claim 1, characterized in that the proportion by weight of panthenol relative to the total weight of the composition ranges from 0.1 to 4%, preferably from 0.4 to 2%.

3. The composition as claimed in claim 1, characterized in that the proportion by weight of lead acetate relative to the total weight of the composition ranges from 0.10 to 0.60%, preferably from 0.25 to 0.60%.

4. The composition as claimed in claim 1, characterized in that the proportion by weight of sulfur relative to the total weight of the composition ranges from 0.10 to 0.50%, preferably from 0.25 to 0.50%.

5. The composition as claimed in claim 1, characterized in that it also comprises at least one other essential oil chosen from a rosemary essential oil, a lavender essential oil, a sage essential oil, and mixtures thereof.

6. The composition as claimed in claim 1, characterized in that it also comprises at least one activator of the oxidation reaction.

7. The composition as claimed in claim 6, characterized in that the activator of the oxidation reaction is chosen from ammonium acetate and ammonium chloride.

8. The composition as claimed in claim 7, characterized in that the activator of the oxidation reaction is ammonium acetate and is present in a proportion by weight relative to the total weight of the composition of approximately 0.10%.

9. The composition as claimed in claim 7, characterized in that the activator of the oxidation reaction is ammonium chloride and is present in a proportion by weight relative to the total weight of the composition of approximately 0.40%.

10. The composition as claimed in claim 1, characterized in that it also comprises at least one agent capable of conferring on the cosmetic composition anti-dandruff and/or anti-seborrheic and/or vitalizing and/or hair-styling properties.

11. The composition as claimed in claim 1, characterized in that it is provided in the form of a lotion, a gel, a milk, a cream or a mousse.
